(19) United States
(12) Patent Application Publication
(10) Pub. No.: US 2015/0272678 A1
(43) Pub. Date: Oct. 1, 2015

(54) LASER SURGICAL INSTRUMENT FOR SPINE SURGERY AND METHOD THEREOF

(71) Applicant: LUTRONIC CORPORATION, Gyeonggi-do (KR)

(72) Inventors: Daniel H. Kim, Houston, TX (US); Kwang Chon Ko, Paju (KR); Hae Lyung Hwang, Seoul (KR)

(73) Assignee: LUTRONIC CORPORATION, Goyang (KR)

(21) Appl. No.: 14/434,393

(22) PCT Filed: Oct. 8, 2013
(86) PCT No.: PCT/KR2013/009008

§ 371 Date: Apr. 8, 2015

Related U.S. Application Data
(60) Provisional application No. 61/710,772, filed on Oct. 8, 2012.

(51) Int. Cl. A61B 18/24

(52) U.S. Cl. CPC A61B 18/24; A61B 2018/00339

(57) ABSTRACT

The present invention relates to a laser surgical instrument for a spine surgery and a method thereof, and supply a spine surgery method which comprises ensuring a path in which a laser surgical instrument enters through a back of a patient, allowing the laser surgical instrument to access an outer surface of an intervertebral disc along the ensured path, allowing the laser surgical instrument to enter an inner side of an epidural space along an outer surface of the intervertebral disc, and irradiating laser to a direction of the intervertebral disc through the laser surgical instrument.

### BACKGROUND OF THE INVENTION

1. Field of the Invention

The present invention relates to a laser surgical instrument for a spine surgery and a method thereof, more particularly, to a surgical laser instrument for a spine surgery and a method thereof for treating lesions occurring on a disc portion of the spine in a minimal invasive manner.

2. Related Art

FIG. 1 is a diagram showing a structure of a human vertebral column. As shown in FIG. 1, a vertebral column 1 is a longitudinal axis of the body, and includes vertebrae connected in a vertical direction and an intervertebral disc provided between the vertebrae.

A vertebral body 11 is formed in a forward direction of each vertebra 10. A vertebra arch including a pedicle 12 and a lamina 13 and a plurality of projections 14 for muscle attachment and joint connection are provided in a backward direction of each vertebra 10. A spine hole 15 forming a vertebral canal is formed between the vertebral body 1 and a vertebra arch. Further, an intervertebral disc 20 serving to adsorb shock is disposed between the vertebral bodies 11. Moreover, a dura mater 40 surrounding a spinal cord 30 passes through an epidural space 50 which is formed at an inner side of the vertebral canal. A nerve bundle is branched from both sides of a dorsal root 60 between the vertebrae 10.

In this case, the intervertebral disc 20 includes an annulus fibrosus 21 provided at an edge of the intervertebral disc 20 formed therein with a fibrocartilage tissue and a seomyuryun 22 which is a gelatin tissue provided at a center portion thereof and strongly coupled with the annulus fibrosus 21 and including a large amount of water. The intervertebral disc 20 has a structure capable of absorbing shock while fixing a vertebral body 11 using resilient characteristics of the fibrosus 21 and the seomyuryun 22.

Lesions such as lumbar herniated intervertebral disc or spinal stenosis occur in the spinal column 1 structure by cause of aging or severe shock of the intervertebral disc 20. The above lesions cause various neurological symptoms such as back pain when the nerve or the nerve roots are pressed or a path through which the nerve or the nerve roots pass become narrow by the intervertebral disc 20.

In order to treat the neurological symptoms, various surgical procedures such as spinal discectomy or fusion procedure of inserting a fusion implant after removing the bone or muscle, have been proposed. However, the surgical techniques according to the related art are limited to a treatment position or treatment lesion or a surgical scale is too large, so that surgical procedures capable of treating a variety of minimally invasive lesions are required.

### SUMMARY OF THE INVENTION

The present invention has been made in an effort to provide a laser surgical instrument for spine surgery capable of treating various lesions in a minimally invasive manner and a surgical operation method thereof.

According to an embodiment of the present invention, there is a provided a spine surgery method including: ensuring a path in which a laser surgical instrument enters through a back of a patient; allowing the laser surgical instrument to access an outer surface of an intervertebral disc along the ensured path; allowing the laser surgical instrument to enter an inner side of an epidural space along an outer surface of the intervertebral disc; and irradiating laser to a direction of the intervertebral disc through the laser surgical instrument.

The allowing of the laser surgical instrument to enter an inner side of an epidural space may include allowing the laser surgical instrument to enter the inner side of the epidural space in a state that an end portion of the laser surgical instrument is bent.

The allowing of the laser surgical instrument to enter an inner side of an epidural space may include maintaining an end portion of an insertion part to be bent in a direction of a back of a patient by 10° to 40° based on a longitudinal direction of the insertion part.

The allowing of the laser surgical instrument to access an outer surface of an intervertebral disc may include allowing the laser surgical instrument in a state that an insertion part of the laser surgical instrument maintains a straight line shape, and the allowing of the laser surgical instrument to enter an inner side of an epidural space comprises: allowing the laser surgical instrument in a state that an end portion of the laser surgical instrument is bent.

The ensuring of the path may include: inserting a needle to an outer surface of the intervertebral disc; and allowing a guide wire to enter an inner side of the intervertebral disc through an outer surface of the intervertebral disc.

The laser surgical instrument may enter the inner side of the intervertebral disc through an outer surface of the intervertebral disc by guide of the guide wire.

The ensured path may be formed in the direction of the intervertebral disc spaced at a location of 4 cm to 14 cm spaced apart from a center line among a surface of the back of the patient. The ensured path may be formed to have an inclined angle of 30° to 65° based on a surface of the back of the patient.

The irradiating of the laser may include irradiating the laser to a direction opposite to a direction in which an end portion of the laser surgical instrument is bent.

The laser surgical instrument may include: a body part; an insertion part extending to a later direction from the body part and including an end portion which is bent; an image part to acquire an image of the end portion of the insertion part; a work channel to form a hollow in a longitudinal direction inside the insertion part; and an irradiating part selectively installed in the insertion part through the work channel and protruding to the end portion of the insertion part to irradiate the laser to a lateral direction.

The spine surgery method may further include a laser blocking part to block the laser irradiated from the laser irradiating part from being irradiated to a direction in which a bent part is bent.

According to another embodiment of the present invention, there is a provided a spine surgery method including: inserting a needle in a direction of an intervertebral disc through a back of a patient; inserting a guide wire to an epidural space of the patient through the needle; inserting an outer sheath using the guide wire; inserting a laser surgical instrument having a bendable end portion into an inner side of the epidural space through an inside of the outer sheath using the guide wire; and irradiating laser in the direction of the intervertebral disc.

According to another embodiment of the present invention, there is a provided spine surgery method including: inserting a needle into an inner side of an intervertebral disc through a back of a patient; inserting a guide wire into the inner side of an intervertebral disc; inserting an outer sheath using the guide wire; inserting a laser surgical instrument into an inner side of the intervertebral disc through an inside of the outer sheath using the guide wire; and irradiating laser to the inner side of the intervertebral disc while bending an end portion of the laser surgical instrument.

### Advantageous Effects

According to the present invention, surgical operation for the lesions present in various locations is possible in the minimally invasive manner and treatment for various types of lesions is possible.

### BRIEF DESCRIPTION OF THE DRAWINGS

FIG. 1 is a diagram showing a structure of a human vertebral column.

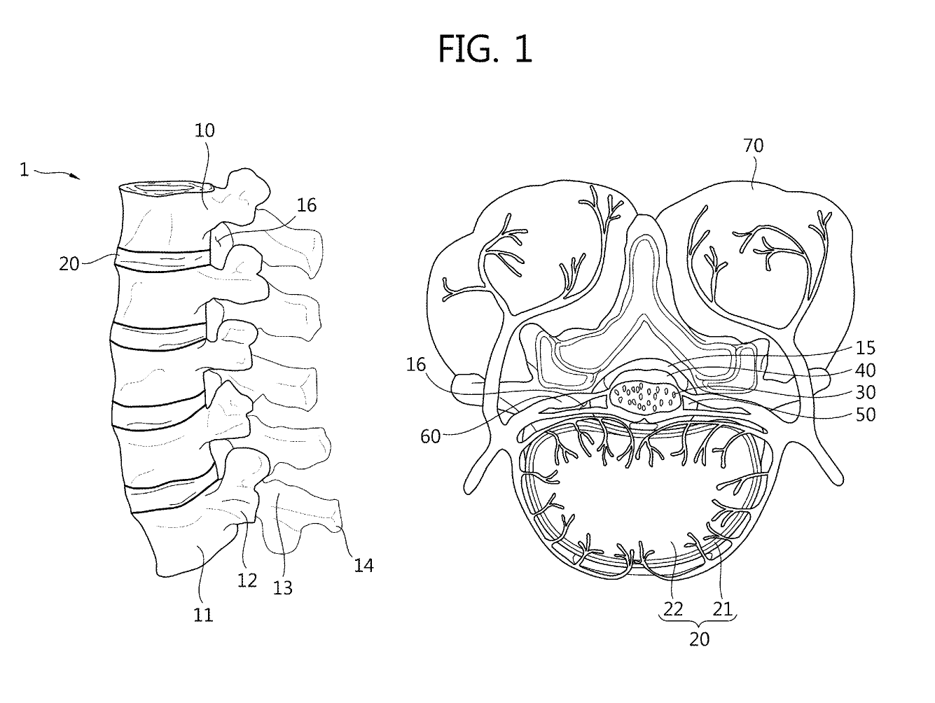

FIG. 2 is a cross-sectional view showing a treatment direction of spinal lesions according to the present invention.

FIG. 3 is a side view showing a laser surgical instrument for spine surgery according to an embodiment of the present invention.

FIG. 4 is a front view showing an end surface of an insertion part of the laser surgical instrument for spine surgery shown in FIG. 3.

FIG. 5 is a cross-sectional view showing an end portion of a laser irradiation unit to be used during spine surgery.

FIG. 6 is a plan view showing an extender to be used during spine surgery.

FIG. 7 is a perspective view showing an end portion of the insertion part of the laser surgical instrument shown in FIG. 3.

FIG. 8 is a perspective view showing another embodiment of FIG. 7.

FIG. 9 is cross-sectional view showing a state of the laser surgical instrument inserted into the surgical position.

FIG. 10 is a flowchart illustrating an example of a surgical method using a laser surgical instrument for the spine surgery.

FIG. 11 is a view showing a surgical tool used in the surgical operation of FIG. 10.

FIG. 12 is a view showing another example of a laser surgical instrument for spine surgery according to the present invention.

FIG. 13 is a view showing another example of a laser surgical instrument for spine surgery and the surgical tool according to the present invention.

FIG. 14 is a flowchart illustrating another example of a surgical operation method using the laser surgical instrument for spine surgery.

### DESCRIPTION OF EXEMPLARY EMBODIMENTS

Hereinafter, a laser surgical instrument for spine surgery and a spine surgery according to an embodiment of the present invention will be described in detail with reference to the accompanying drawings. Such a position of the constituent elements has been described with reference to the drawings. The structure of the present invention may be exaggerated, omitted or schematically drawn for the purpose of convenience or clarity. In addition, the size of elements does not utterly reflect an actual size. Accordingly, the present invention is not limited thereto, but various devices may be added, changed, or omitted to the above components according to requirements.

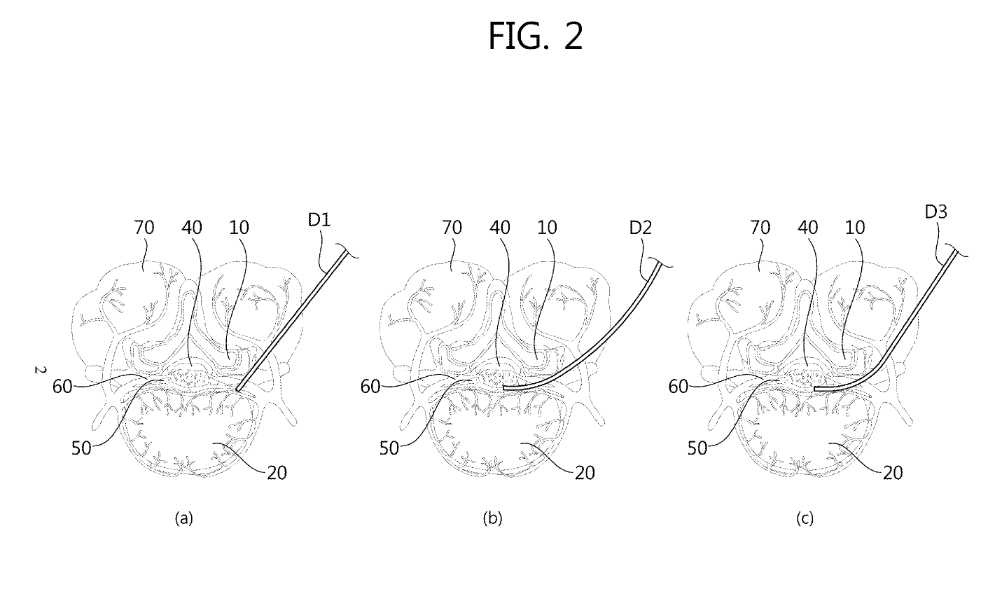

FIG. 2 is a cross-sectional view showing a treatment direction of spinal lesions according to the present invention. As shown in FIG. 2, an intervertebral disc 20 is disposed between a vertebra and a vertebra which are vertically close to each other in the front of the vertebral column. Further, a spinal cord 30 and a dura mater 40 surrounding the spinal cord 30 pass through an epidural space 50 which is vertically formed in a backward direction of the vertebral column. A nerve branched from the dura mater 40 extends to an outer side of the vertebral column through a foraminal 16 formed on both sides between the vertebra and the vertebra. Moreover, a back muscle 70 is arranged at a rear of the spine.

A lumbar herniated intervertebral disc being one of the major spinal lesions occurs when the intervertebral disc 20 protrudes toward a rear side of a vertebral column due to aging or serve shock to press spinal nerves. Further, a spinal stenosis spinal canal or the foraminal becomes narrow to press the nerves. Accordingly, in order to treat the above spinal lesions, the protruded portion of the intervertebral disc is removed by inserting the surgical device from the back of the patient (upper side in FIG. 2) to the spine direction or a space may be ensured by removing the microstructure between the spinal canal or the foraminal. In this case, the manner of entering the surgical instrument may take into consideration a plurality of directions.

First, as shown in FIG. 2, by using a surgical instrument including the insert part D1 having a straight linear shape, the manner of entering the surgical instrument into the treatment position along a straight path may be considered. Thus, in the case of using the surgical instrument having an insertion part having a straight line shape, it is possible to access the surgical instrument to an exposed portion of an outer side of the vertebral column among the intervertebral disc 20, and it is impossible to access the surgical instrument to a rear portion of the intervertebral disc 20, that is, a part exposed to an epidural side through the foraminal 16. In this case, when the surgical instrument having an insertion part having a straight line shape is used, since an access position is limited, it is difficult to treat lesions such as a symptom in which a rear side of the intervertebral disc 20 protrudes or the spinal stenosis symptom between the overhanging.

Meanwhile, as shown in FIG. 2b, the manner of entering the surgical instrument including an insertion part D2 having a straight line shape into the treatment position along a straight path may be considered. As described above, when the surgical instrument including an insertion part D2 having a straight line shape is used, it is possible to access the surgical instrument to a part exposed to an outer side of the vertebral column among the intervertebral disc 20 and an inside of the epidural through the foraminal 16. Accordingly, it is possible to surgically operate various lesions occurring at a rear side of the intervertebral disc 20.

Further, as shown in FIG. 2c, a manner of entering the surgical instrument including an insertion part D3 having a body having a straight line shape and a bent end into the treatment position may be considered. Accordingly, it is possible to enter the surgical instrument into the epidural space 50 through a foraminal 16 of the vertebral column using the bent end structure. Accordingly, access to an outer side and a rear side of the intervertebral disc 20 is possible so that there is no limitation on the treatment position and treatment with respect to various Lesions. In particularly, the structure having an insertion part having a curved shape shown in FIG. 2(b) is manufactured to be difficult and a necessary curved shape is different according to locking and treatment position of a patient. In contrast, the scheme of FIG. 2c may be used in various patients and various treatment positions using the surgical instrument of the same structure.

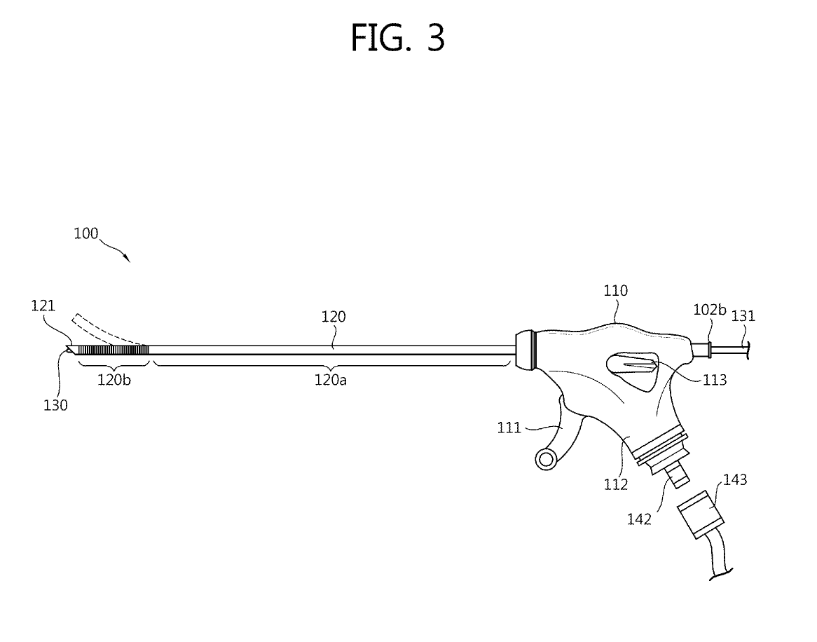

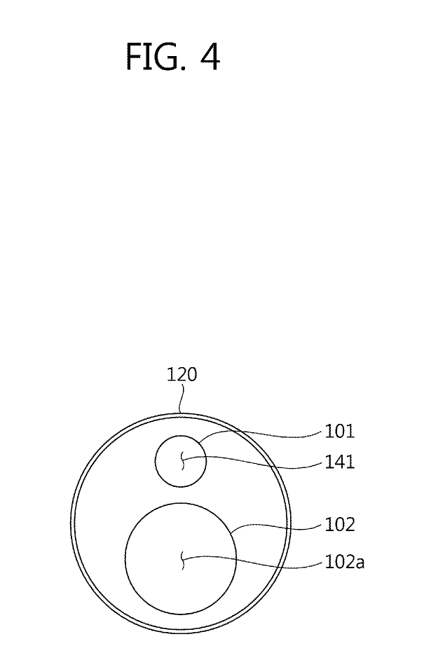

Hereinafter, the laser surgical instrument 100 for spinal surgery that can proceed with the surgical operation as shown in FIG. 2c will be described in detail with reference to the drawings. FIG. 3 is a side view showing a laser surgical instrument for spine surgery according to an embodiment of the present invention, and FIG. 4 is a front view showing an end surface of an insertion part of the laser surgical instrument for spine surgery shown in FIG. 3.

The laser surgical instrument 100 according to an embodiment of the present invention includes: an insertion part 120 inserted into a body during surgical operation, a body part 110 in which the insertion part 120 is installed, and a laser irradiating part 130 to irradiate laser to a surgical region through the insertion part 120.

First, the insertion part 120 is connected to the body part 110, and extends to one side direction from the body part 110. Moreover, the insertion part 120 has a long pipe structure having a narrow section so that minimal invasive surgery is possible in a body during surgical operation. In addition, the insertion part 120 may be configured by at least two regions including a straight line part 120a and a bent part 120b in a longitudinal direction.

The straight line part 120a is disposed at a part connected to a body and forms a body of the insertion part 120. The straight line part 120a has a straight line shape extending straightly and is made of a rigid material. Accordingly, when the insertion part 120 is inserted through the body's tissues during surgery, it is possible to access the surgical location without changing the shape.

The bent part 120b is formed at an end portion of the straight line part 120a, and may be selectively bent. In detail, the bent 120b may be configured by a plurality of joint members, and may be bent in a preset direction by a user's operation. The joint members constituting the bent part 120b are made of rigid materials. Accordingly, during the surgical operation, the insertion part is inserted through a body issue, it is possible to access the surgical instrument to a surgical operation position without changing the shape in a bent state or a state which is not bent.

Meanwhile, a grip member 112 which the user may grip during the surgical operation is provided at the body part 110. An operating member 111 to selectively bend the bent part 120b is provided at a front end portion of the grip member 112. The operating member 111 shown in FIG. 3 is configured to have a shape such as a trigger of the gun. If the operation member 111 is pulled, the bending angle of the bent portion (120b) is increased. In this case, the maximum angle at which the bent portion 120b is bent by the operation of the operation member 111 may be configured to form within 45° by taking into consideration the surgical operation path and a position of foraminal.

Here, the bent part 120b and the operation member 111 for bending the bending part 111 may be configured to be operated in connection with each other by the wire provided in the interior. In addition, it is also possible to apply the known bending interlocking structure in the prior art. Further, the operation member 111 having a shape such as a trigger of the gun according to the embodiment is illustrative purpose only and various types of operation member may be configured. In this case, the bent part 120b may be configured to be rotatable in both directions of the straight line part 120a relative to the upper and lower sides (in FIG. 2). However, in the present embodiment, the bent part 120b may be bent in an upward direction based on the straight line part 120a. In general, spinal surgery is performed through a back of the patient in a state that the patient lays on the patient's face. Therefore, when considering a path of inserting the insertion part 120 to the epidural through the foraminal from the inserted position, it is possible to reach the surgical operation position inside the epidural by configured such that the bent part is bent in a upward direction (that is, a direction opposite to a direction in which the grip portion of the body part is formed based on the straight line part).

On the other hand, a locking member 113 for maintaining a state in which the bent part 120b is bent may be provided at one side of the body part 110. Therefore, when the bent part 120b is bent at an angle suitable for the surgical operation in the surgical operation position, it is possible to stably perform the surgical operation in a state where the bent part maintains the bent shape using a locking member 113. The locking member 113 may be configured in such a way that an operation member 111 for operating the bent part 120b is not operated by using the configuration of the latch, or a linkage device for transmitting a driving force to the bent part from the operation member 111 is not operated.

As shown in FIG. 4, the at least two channels are formed inside the insertion part 120. One of the channels may be an optic channel 101, and one channel may be a working channel 102.

The optic channel 101 forms an optical path of an image part. The image part is a configuration for acquiring an image of an end portion of the insertion portion 120, and it is possible to obtain a position of an end portion of the insertion portion 120 and an image of a scene of the surgical operation during the surgical operation. A lens 141 is provided at the end portion of the optic channel 101. Light reflected from the patient's tissue passes through the lens 141 and then is transmitted through the optic channel 101. The optic channel 101 according to the present embodiment extends to an optic connector 142 installed at one side of the body part through the insertion part 120 and the body part 110. Therefore, an image cable 143 connected to a separate image processor (not shown) is connected to the optic connector 142, and images the image cable 143 to an image device (not shown) to obtain an image an end portion of the insertion part 120.

The working channel 101 forms the hollow path as a channel for inserting various surgical tools required during the surgical operation. Therefore, the laser irradiation part 130, an expander 150 such as an inflatable member, various surgical tools such as forceps member (not shown) may reach the surgical operation position through the working channel 101. In the present embodiment, the working channel 101 is formed through the insertion part 120 and the body part 110. An input hole 102b is formed at an rear end of the body portion 110, and an output hole 102a is formed in the end portion of the insertion part 102a.

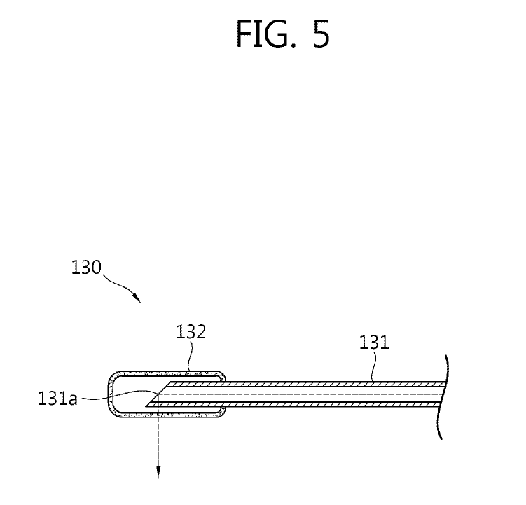

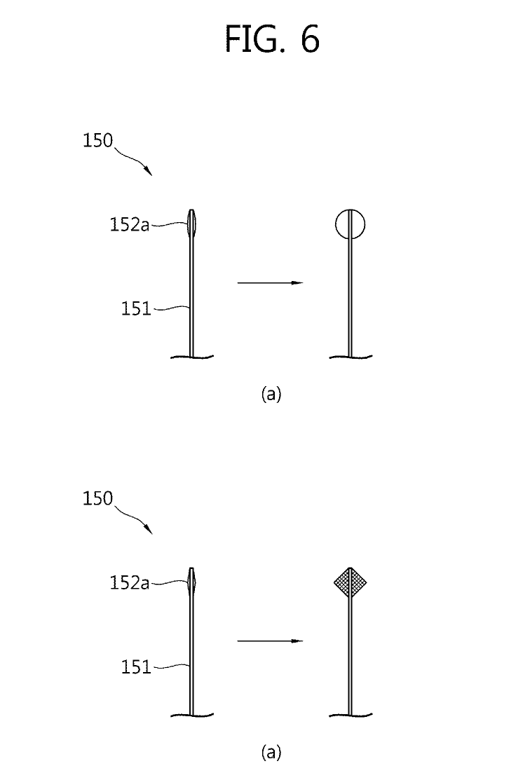

FIG. 5 is a cross-sectional view showing an end portion of a laser irradiation unit to be used during spine surgery, and FIG. 6 is a plan view showing a spine extension to be used during spine surgery. Hereinafter, the various tools inserted through the working channel and used during the surgical operation will be described in detail with respect to with reference to FIGS. 5 and 6.

As shown in FIG. 5, the laser irradiation part 130 is a device which transfers the laser generated from a laser light source (not shown) to the surgical operation location. The laser irradiation part 130 is installed so as to protrude to the end portion of the insertion part 120 via the above-mentioned work channel 101, and irradiates the laser to the surgical site during the surgical operation so that the treatment proceeds. The laser irradiation part 130 is configured to include an optical fiber 131 for transferring the laser and a cover member 132 installed at an end portion of the optical fiber 131.

In this case, the optical fiber 131 forms a path through which the laser passes. One end of the optical fiber 131 is connected to the laser light source (not shown) side, and the laser transferred from the laser light source is irradiated to the surgical site through the other end of the optical fiber 131. As shown in FIG. 5, a surface of the other end of the optical fiber 131 forms an inclined surface 131a, and a side of the other end is pointed. Thus, the laser transferred through the optical fiber 131 is reflected by the refractive index difference in the inclined surface 131a and is irradiated toward the lateral direction.

Meanwhile, the cover member 132 surrounds an end portion of the optical fiber 131 to which the laser is irradiated from the optical fiber 131. This is for the purpose of preventing reflection characteristics of the laser in the slope surface from being changed according to the characteristics of a material making contact with the inclined surface of the optical fiber 131 because an end portion of the optical fiber 131 is exposed to the outside. The cover member 132 is composed of a transparent material, and the laser irradiated from the optical fiber 131 passes through the cover member 132 and is irradiated to a surgical site.

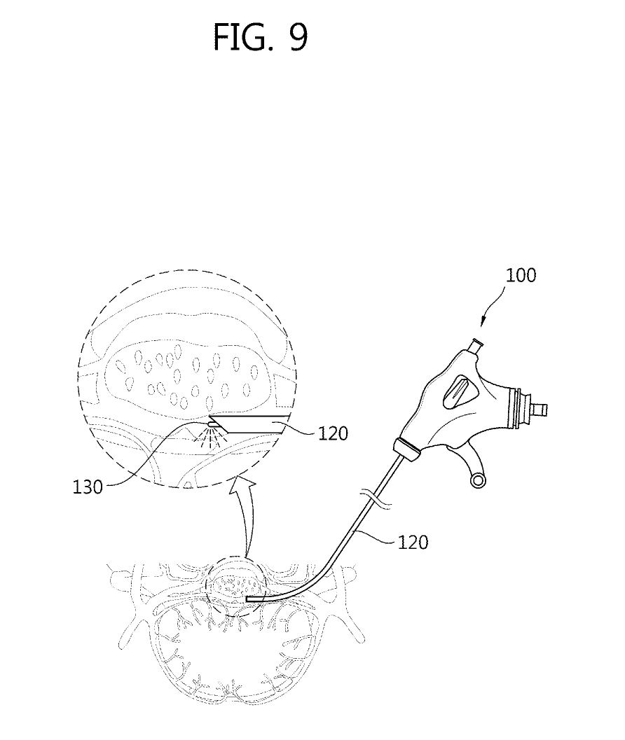

In this way, the laser irradiation part 130 is configured to irradiate the laser in one direction from the end portion of the insertion part 120. Therefore, the laser irradiation part 130 may be installed to irradiate the laser in a direction opposite to the direction in which the insertion part is bent (see FIG. 9). More specifically, the laser irradiating part may be installed so that a pointed portion of the end portion of the optical fiber 131 is disposed in a direction opposite to the bent direction of the insertion part, that is, a direction of the disk. In this case, it is possible to treat spinal lesions by irradiating the laser in the direction of the intervertebral disc 20 instead of a direction of the dura mater 40 in a state that the laser irradiating part 130 enters at an inner side of the epidural space 50.

Meanwhile, the extender 150 shown in FIG. 6 is a configuration to extend an internal space of the tissue by applying a mechanical force in the internal space inside the human body tissue during the surgical operation. The extender 150 includes a body 151 such as a wire having an elongated member shape and a selectively inflatable balloon member 152a provided at an end portion thereof. The extender 150 is inserted through a work channel 101 of the insertion part as in the laser irradiation part 130. The balloon member 152a may be selectively expanded using a flow path connected to the balloon member 152a along the body 151. When the balloon member 152a protrudes to be inflated to an end portion of the insertion part 120, the balloon member 152a applies a mechanical force on the adjacent tissue, while extend portioning the surgical operation space.

The extender 150 may be configured to be inserted together with a work channel 101 in which the laser irradiating part 130 is inserted. The extender 150 may be configured to be inserted into a separate channel from the work channel 101 in which the irradiating part 130 is inserted. Thus, upon irradiating the laser or before irradiating the laser, it is possible to ensure a treatment space in a state that the extender 150 is disposed forward of the end portion of the laser irradiation part.

Therefore, the laser is irradiated to a position adjacent to the surgical site to prevent from being damaged. Additionally, it is also possible to perform a function to block the laser from being irradiated to the direction of a dura mater side while maintaining the extended state. Furthermore, the extender 150 applies mechanical strength to the adjacent tissue while being expanded, which may contribute to the treatment of lesions such as spinal stenosis.

The present embodiment describes the extender 150 including a balloon member 152a at an end thereof as an example. However, in addition, as shown in FIG. 6b, it is possible to ensure a treatment space by providing a basket member 152b which may be selectively deployed at an end portion thereof, and the extender 150 may be variously configured to ensure the treatment space by applying a mechanical force to the tissue.

Meanwhile, although the present embodiment has been described with respect to the laser irradiation part and the extender of the various tools to be used during spinal surgery, various tools such as forceps for detaching the tissue and the tubes for injecting drugs may be inserted and used through the working channel.

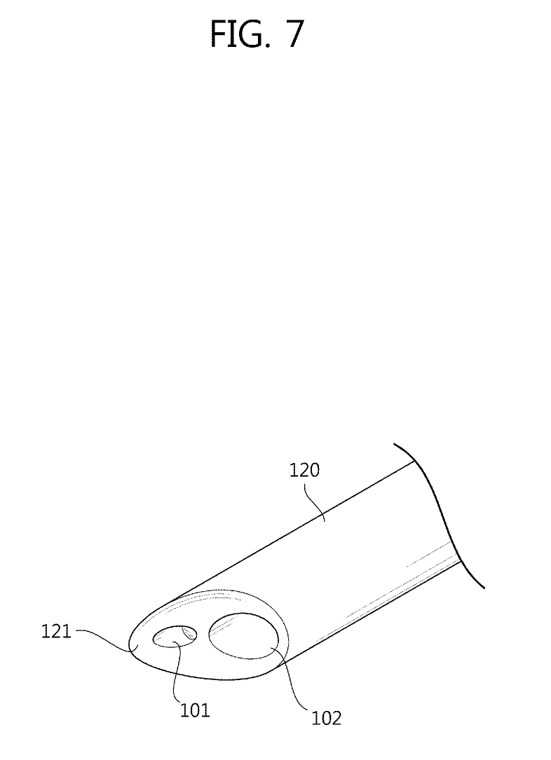

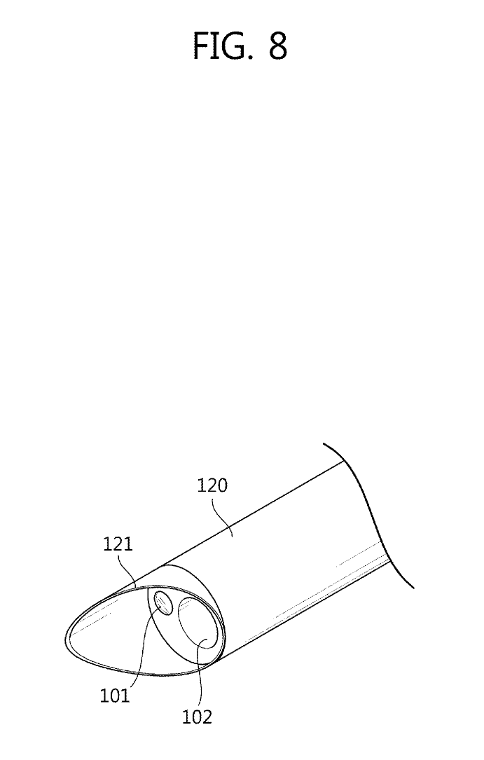

FIG. 7 is a perspective view showing an end portion of the insertion part of the laser surgical instrument shown in FIG. 3, and FIG. 8 is a perspective view showing another embodiment of FIG. 7. At the end portion of the insertion part 120 according to this embodiment the laser blocking part 121 may be formed. As described above, the laser irradiation part 130 irradiates the laser in the direction of the intervertebral disc 20 to protrude to the end portion of the insertion part 120. However, when the laser irradiating part is installed in a wrong direction or an end portion of the irradiating part is broken, the laser is irradiated to the dural direction to damage a nerve tissue such as the spinal cord. Therefore, in the present embodiment, a laser blocking part 121 may be formed at an end portion of the insertion part 120 to block the laser from being irradiated toward the dura mater 40 side.

As shown in FIG. 7 and FIG. 8, a laser blocking part 121 is formed at an end portion of the insertion part 120, and protrudes more than the adjacent end surface in a longitudinal direction. The laser blocking part 121 may be installed in a direction in which the dura mater tissue is located during the surgical operation, that is, a direction in which the bent part 120b of the insertion part is bent.

As shown in FIG. 7, a laser blocking part 121 may be provided in such a way to form a protruding structure of the shape of the end portion itself of the insertion portion 120. As illustrated in FIG. 8, the laser blocking part 121 may be configured as a separate cap member which is fastened to the end portion of the insertion part.

FIG. 9 is cross-sectional view showing a state of the laser surgical instrument inserted into the operative position. In the above mentioned laser surgical instrument 100, since the end portion of the insertion part 120 may be bent, the laser surgical instrument 100 may be inserted from a back of a patient and enter to the inside of the epidural space 50 through the foraminal 16. The surgical operation may be performed by irradiating the laser toward the intervertebral disc 20 using the laser irradiating part 130 installed in the work channel 101. In this case, a position to which the laser is irradiated in a longitudinal direction of the spinal column may be changed by rotating the body part 110 of the laser surgical instrument by −90° to 90° by using a straight line portion of the insertion part 120 as the rotation axis. Therefore, it is possible for a variety of surgical positions. Furthermore, it is also possible to treat lesions such as spinal stenosis by adjusting the bending angle of the insertion part in the state that the insertion part 120 is inserted. As such, the present invention provides the laser surgical instrument 100 capable of treating various lesions for various locations of a spine organ.

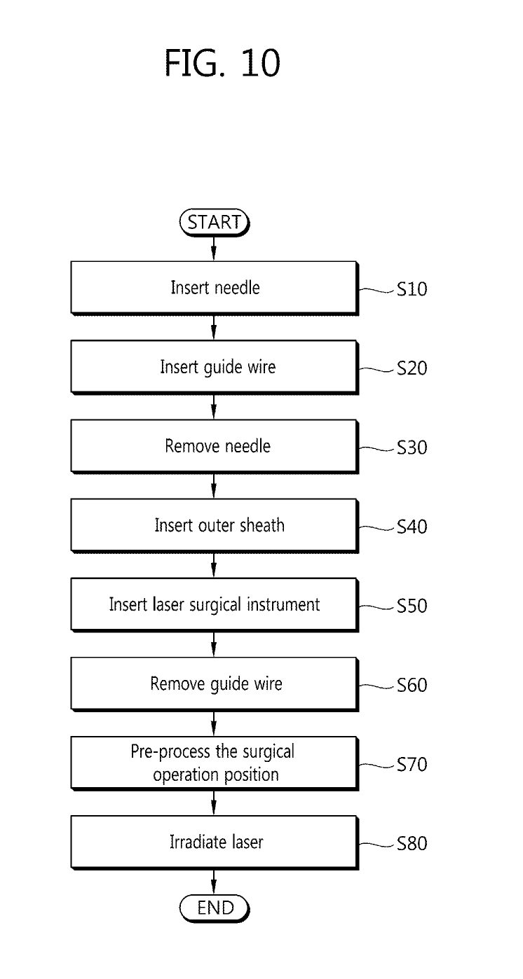

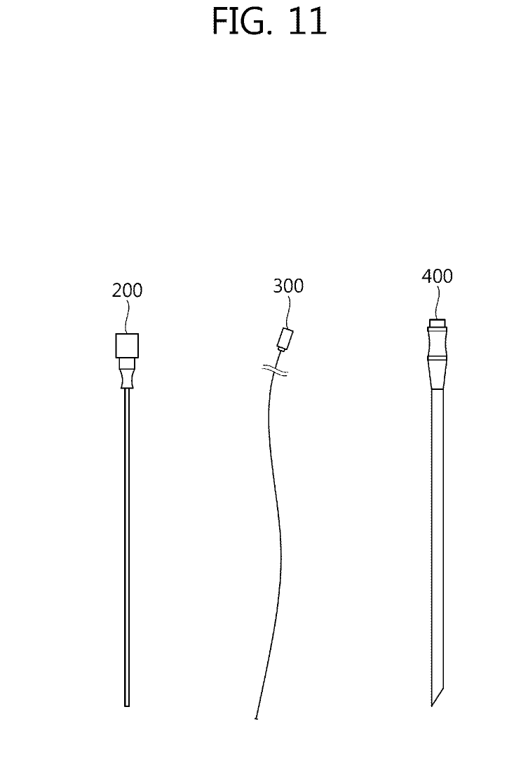

Hereinafter, an example of the surgical operation method using a laser surgical instrument for the spine surgery according to the present embodiment will be described in detail with reference to the accompanying drawings. FIG. 10 is a flowchart illustrating an example of a surgical method using a laser surgical instrument for the spine surgery, and FIG. 11 is a view showing a surgical tool used in the surgical operation of FIG. 10.

The surgical operation method according to the present embodiment may be performed using separate surgical tools such as a needle 200, a guide wire 300 and an outer sheath 400 shown in FIG. 11 in addition to the laser surgical device.

In this case, the needle 200 and the guide wire 300 are a configuration to ensure a path in which the surgical instrument 100 enters a surgical site. In detail, the needle may use a tuohy needle. The present embodiment may use a needle having a length of 4˜5 inches and having a straight line shape being gauge 14 or 15. Further, the guide wire 300 is a wire member having predetermined rigidity and has a diameter of 1 mm or less. In detail, the guide wire 300 may use a wire capable of being inserted in a hollow interior inside the needle 200 using the wire being a gauge 20.

Further, the outer sheath 400 is a member for forming a space in which the laser surgical instrument is inserted when the surgical operation is performed. The outer sheath 400 may use a hollow member having a straight line shape which has an outer diameter of 8 mm or less, and the inner diameter of 2.5 mm. The present embodiment may use an outer sheath 400 having the outer diameter of 4.0 mm and the internal diameter of 3.75 mm. The outer sheath is formed therein with a slope surface having an end portion pointed to a lateral side to facilitate the entry inside the human body. In the present embodiment, the outer sheath may be configured to form an angle of 30° to 45° in the longitudinal direction.

However, the above surgical tools may be variously changed and used depending on the physical condition and the treatment position of the patient, and is possible to be substituted by a different tool to perform the functions described in the present embodiment.

The spinal surgery method according to the present embodiment may be performed by starting a step of ensuring a path in which the laser surgical instrument may enter using the needle 200 and the guide wire 300. In the present embodiment, the spinal surgery method may be performed in a state that the patient lies down to expose the back, and may be performed to ensure a path from the surface of the back to the spine direction.

As shown in FIG. 10, a laser surgical instrument firstly inserts a needle (S10). The needle 200 may be inserted into a lateral side spaced from a center line of a back of a patient among a surface of the back of the patient (upward and downward directions of a human body in which the vertebral column is formed). The distance may be in the range of 4 cm to 14 cm spaced apart from the center line. In the present embodiment, the needle 200 may be inserted at a location of 10 cm to 12 cm spaced apart from the center line. The needle 200 is inserted into the direction of the intervertebral disc 20. The needle 200 is inserted into the tilted angle of 30° to 65° relative to the surface of a back of the patient. In the present embodiment, the needle 200 may be inserted into the tilted angle of 45° to 60°. Since the needle 200 uses the tuohy needle which is the aforementioned straight member, it is difficult to enter the needle the epidural space 50. However, since the needle 200 is inserted along an inclined path, the needle 200 may reach the outer surface close to the epidural space among the rear side of the intervertebral disc.

When the needle 200 reaches the outer surface of the intervertebral disc 20, a guide wire 300 is inserted (S20). The guide wire 300 is inserted through a hollow interior of the needle 200. When the end portion of the guide wire 300 reaches the outer surface of the intervertebral disc 20 by inserting a guide wire 300 along the hollow interior of the needle 300, the guide wire 300 may be further inserted by a predetermined length. Therefore, the end portion of the guide wire 300 travels along the outer surface of the intervertebral disc 20 and enters into the epidural space 50 through the foraminal 16.

Since such steps are possible to determine a position of the end portion of the needle 200 and the position of the end portion of the guide wire 300 in real time using radiographic photograph, it is possible to proceed with the surgical operation by using an image that is provided.

Further, although the present embodiment has been described a method for entering the guide wire the epidural space using one tuohy needle, it is possible to further induce the entry route of the guide wire using a plurality of needles. For example, as described above, in a state of inserting a straight needle being a gauge 14 or 15, the guide wire can easily enter an inside of the epidural space by inserting a curved needle having the gauge 18 or 20 and an end portion having a curved shape into the inside of the straight needle, and inserting the guide wire into the hollow interior of the curved needle.

Next, when the guide wire 300 is inserted into the epidural space 50, the needle 200 is removed (S30), and the step of inserting the outer sheath 400 is performed (S40). The outer sheath 400 has a diameter greater than that of the needle 200 and that of the guide wire 300. Thus, after additionally cutting the adjacent tissue of the position in which the guide wire 300 is inserted, the outer sheath 400 may be inserted. Alternatively, after cutting the adjacent tissue of the position, it is possible to ensure the insertion path of the outer sheath 400 using a separate scalpel and dilator.

Since the outer sheath 400 is composed of a linear member as in the needle in this step, the outer sheath 400 reaches the outer peripheral surface of the intervertebral disc. Further, a space in which various surgical tools for processing subsequent surgical operation are inserted is formed inside the outer sheath.

If the location of the outer sheath 400 is fixed, the laser surgical instrument 100 is inserted into the outer sheath (S50). As described above, the laser surgical instrument 100 includes a working channel 101 having a hollow interior. After inserting the guide wire 300 into the work channel 101, the guide wire 300 may be inserted to the surgical location of the guide wire 300.

In this case, the laser surgical instrument 100 may maintain the insertion part 120 in a straight line until it reaches the outer surface of the intervertebral disc 20. Further, when the end portion of the insertion part 120 reaches near the outer surface of the intervertebral disc 20, the end portion of the insertion part 120 is bent toward a surface of a back (upward direction when the patient lies down to expose the back) so that the surgical operation may be performed along the guide wire 300. In this case, the end portion of the insertion part 120 in the laser surgical instrument 100 may pass through the foraminal 16 and reach the surgical operation position inside the epidural space 50.

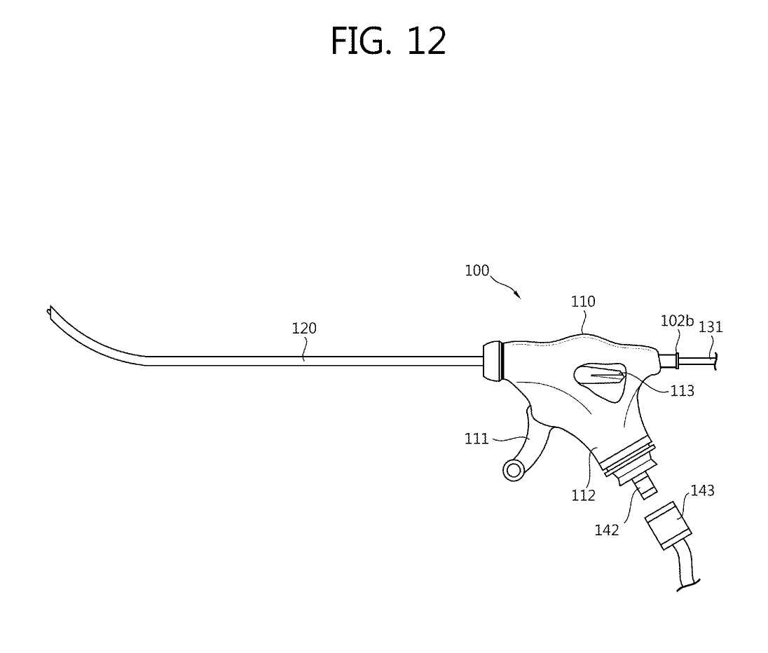

The spinal surgery method according to the present embodiment describes a method of performing a surgical operation using the laser surgical instrument such that the end portion of the insertion part 120 is selectively bent. However, as shown in FIG. 12, it is possible to perform the surgical operation using the laser surgical instrument 100 including an end portion of the insertion part 120 which is bent to a lateral side. In this case, even if using the needle 200 of the straight line and the outer sheath 400, since the inner diameter of the outer shear 400 is larger than the diameter of the insertion part 120 of the laser surgery unit 100, an end portion of the insertion part 120 is inserted into the outer surface of the intervertebral disc 20 through the outer sheath 400 is inserted in a straight line and that, by adjusting the angle it is possible to insert to enter the epidural 50 through the foraminal 16.

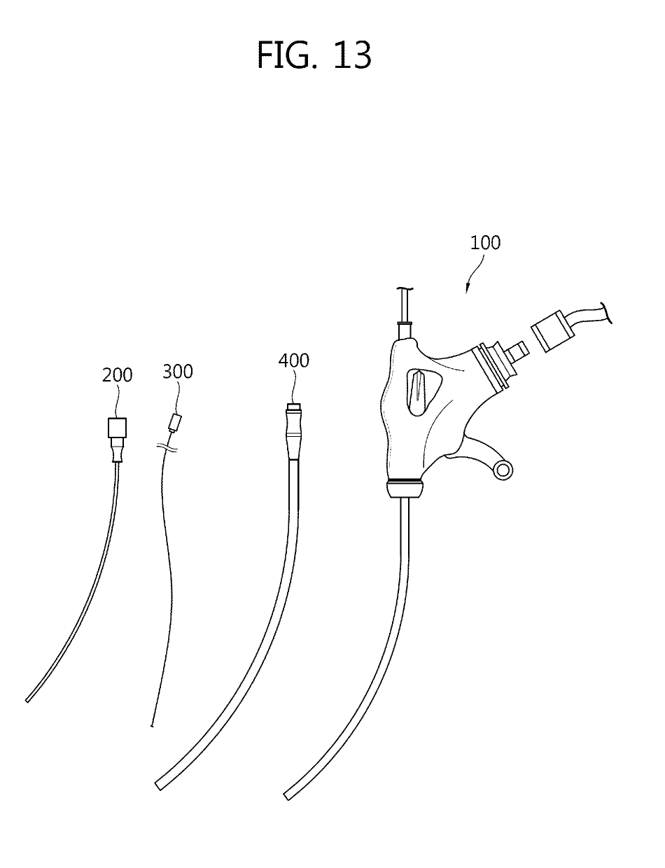

In addition, as shown in FIG. 13, after the entry route of the curve shape is ensured using a needle 200 having a curved structure, the surgical operation may be performed using the laser surgical instrument 100 including the outer sheath 400 having a curved shape and the insertion part having the curved shape. In this case, since the laser surgical instrument 100 enters along a curved path from the surface of a back of a patient, the end portion of the insertion part of the laser surgical instrument may reach the inside the epidural space.

On the other hand, the laser surgical instrument reaches the surgical position in the above step, the guide wire is removed (S60). Next, Then, prior to performing the surgical operation using the laser, it is possible to perform pre-processing step for forming an excellent environment of the surgical operation position (S70).

In this step, the forceps member is inserted through the work channel 101 of the laser surgical instrument 100 to remove various tissues such as a fat present in the surgical operation position, thereby ensuring the accessibility and visibility of the surgical operation position. Alternatively, a space to performing the surgical operation may be ensured by inserting an extender 150 through the working channel 101 of the laser surgical instrument 100. In addition, various pre-processing operations for performing excellent surgical operation using various surgical tools through the working channel may be performed.

After that, the laser surgical instrument irradiates the laser to the surgical operation position (S80).

In this step, a laser irradiation part is inserted through the working channel 101 of the laser surgical instrument 100, and the end portion of the laser irradiation part 130 may be installed to protrude to an end portion of the insertion part 120. Here, as described above, the laser irradiation part 130 is configured to irradiate the light in one direction rather than in the front direction. Therefore, the intervertebral disc 20 may be installed in the working channel 101 so that the laser irradiation part 130 may irradiate light to a direction opposite to a direction in which the insertion part is bent.

Moreover, the laser in this step may be output and irradiated to the laser surgical location. In this case, the laser may use Nd:Yag laser having wavelength of 1414 nm. various procedures of coagulating or cutting a tissue of the surgical location by changing the pulse characteristics of the laser to apply energy tissue of the surgical location may be performed.

In this case, as described above, since the laser blocking unit 121 for blocking the laser from being irradiated to a dural direction is disposed at the end portion of the insertion part of the laser surgical device 100, it is possible to stably perform the surgical operation. When the surgical operation is performed in a state that the above-described extender 150 and the laser irradiation part 130 are simultaneously inserted, a stable surgical operation space may be ensured and the laser may be prevented from being irradiated to the dural direction.

In the present step, the user may adjust an inserting depth of the laser surgical instrument 100 or adjust an irradiated position of the light within a predetermined range by rotating a direction of a steering wheel (right and left directions of the patient during depth control of the surgical instrument, and upward and downward directions of the patient when the direction of the steering wheel is rotated). Accordingly, various spinal lesions such as a lumbar herniated intervertebral disc or a spinal stenosis in the user may be surgically treated in a minimally invasive manner using the laser surgical instrument for spine surgery.

Although the above embodiment has illustrated a method of surgically treating lesions occurring at an outer side of the intervertebral disc, various lesions may be treated using the laser surgical instrument according to the present invention.

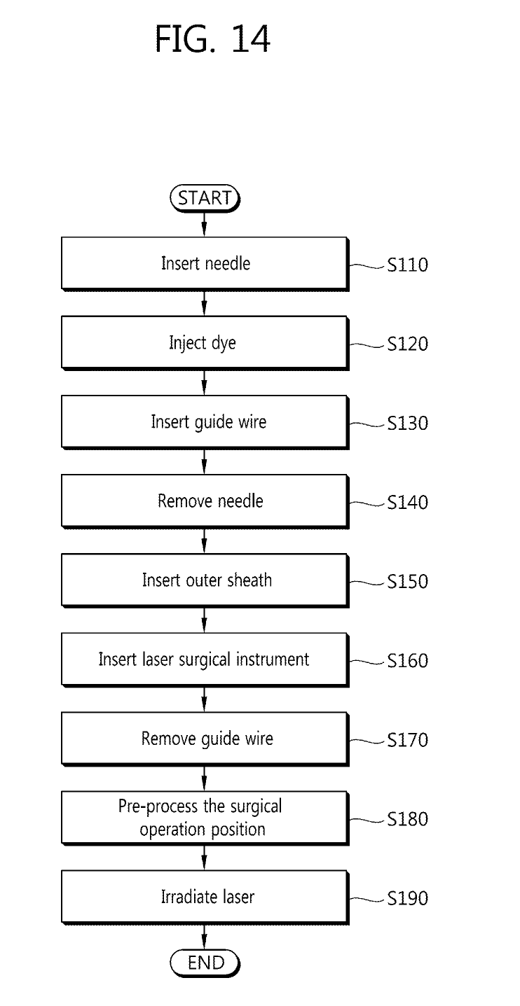

Hereinafter, another example of a surgical operation method using the laser surgical instrument for spine surgery will be described with reference to the accompanying drawings. FIG. 14 is a flowchart illustrating another example of a surgical operation method using the laser surgical instrument for spine surgery.

The above-described method is directed to a method of surgically treating lesions present in the inner side of the intervertebral disc as compared with an existing method of surgically treating lesions preset in an outer side of the intervertebral disc. However, the configurations and the steps similar to those of the above surgical operation method are omitted to avoid repetition.

As shown in FIG. 14, a laser surgical instrument firstly inserts a needle (S110). In the same manner as in the above embodiment, in a state that the patient lies down to expose the back in upward direction, a needle 200 may be inserted in a direction of the intervertebral disc 20 through the surface of the back of the patient.

However, in the above-described embodiment, if it is detected that the end portion of the needle 200 arrives at an outer surface of the intervertebral disc 20, the insertion of the needle stops. In contrast, in the present embodiment, if it detected that the needle 200 arrived at the intervertebral disc 20, the end portion of the needle 200 may entered to a depth of 4 mm to 5 mm inside of the intervertebral disc 20 by additionally inserting the needle 200.

Next, a dye is injected into an inside of the intervertebral disc 20 (S120). The dye may use a substance to dye the intervertebral disc tissue stained in blue, and the dye may be injected by using the inserted needle 200. Accordingly, the intervertebral disc tissue is dyed in blue, which is possible to determine more clearly the position of the intervertebral disc 20 using the radiation imaging apparatus which is photographed during surgery.

When the dye is injected, a guide wire 300 is inserted (S130). The guide wire 300 may be inserted through a hollow interior of the needle 200. The guide wire 300 may be inserted so that the end portion of the guide wire 300 enter an inside of the intervertebral disc 20.

Next, in the same manner as in the surgical method of the above embodiment, after the needle is removed (S140), and the outer sheath 400 may be inserted by additionally cutting a tissue of the patient (S150). In this case, the outer sheath may be inserted to an outer surface of the intervertebral disc 20, and may be inserted into an inside of the intervertebral disc 20 as in the needle 200 and the guide wire 300.

If the outer sheath 400 is inserted, the laser surgical instrument 100 is inserted into the outer sheath 400 (S160). In this case, the laser surgical instrument 100 is inserted into a surgical operation position inside the intervertebral disc 20 by guide of the guide wire 300 in the same manner as in the surgical method of the above embodiment. In this case, the laser surgical instrument may be inserted by detecting a color of the tissue photographed by an image unit of the laser surgical instrument while confirming an insertion position.

If the end portion of the laser surgical instrument 100 is inserted into an inside of the intervertebral disc 20 (S170). It is possible to perform pre-processing step with respect to the surgical operation position (S180)

Next, the laser irradiating part 130 is installed at the laser surgical instrument 100 and irradiates the laser to a tissue inside the intervertebral disc 20 so that the surgical operation is performed. In step S190, the laser surgical instrument may adjust a bent angle of the end portion of the insertion part 120 or adjust an irradiated direction of the laser while rotating a handle direction of the laser surgical instrument. Accordingly, in a state that an outer surface of the intervertebral disc 20 is penetrated once, various internal positions may be surgically operated.

As described above, by using the laser surgical instrument including a bent end portion, various types of lesions with respect to position of various lesions locations may be surgically operated in the minimally invasive manner. However, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A spine surgery method comprising:
ensuring a path in which a laser surgical instrument enters through a back of a patient;
allowing the laser surgical instrument to access an outer surface of an intervertebral disc along the ensured path;
allowing the laser surgical instrument to enter an inner side of an epidural space along an outer surface of the intervertebral disc; and
irradiating laser to a direction of the intervertebral disc through the laser surgical instrument.

2. The spine surgery method of claim 1, wherein in the step for allowing the laser surgical instrument to enter the inner side of the epidural space, the laser surgical instrument is allowed to enter the inner side of the epidural space in a state that an end portion of the laser surgical instrument is bent.

3. The spine surgery method of claim 2, wherein in the step for allowing the laser surgical instrument to enter the inner side of an epidural space, the laser surgical instrument maintains an end portion of an insertion part to be bent in a direction of a back of a patient by 10° to 40° based on a longitudinal direction of the insertion part.

4. The spine surgery method of claim 1, wherein the step for allowing the laser surgical instrument to access the outer surface of the intervertebral disc is performed in a state that an insertion part of the laser surgical instrument maintains a straight line shape, and
the step for allowing the laser surgical instrument to enter the inner side of the epidural space comprises is performed in a state that an end portion of the laser surgical instrument is bent.

5. The spine surgery method of claim 1, wherein the step for ensuring the path comprises:
inserting a needle to the outer surface of the intervertebral disc; and
allowing a guide wire to enter the inner side of the intervertebral disc through the outer surface of the intervertebral disc.

6. The spine surgery method of claim 5, wherein the laser surgical instrument enters the inner side of the intervertebral disc through the outer surface of the intervertebral disc by guide of the guide wire.

7. The spine surgery method of claim 1, wherein the ensured path is formed from a location of 4 cm to 14 cm spaced apart from a center line among a surface of the back of the patient, to the direction of the intervertebral disc spaced.

8. The spine surgery method of claim 1, wherein the ensured path is formed to have an inclined angle of 30° to 65° based on a surface of the back of the patient.

9. The spine surgery method of claim 1, wherein in the step for irradiating the laser, the laser is irradiated to a direction opposite to a direction in which an end portion of the laser surgical instrument is bent.

10. The spine surgery method of claim 1, wherein the laser surgical instrument comprises:
a body part;
an insertion part extending to one direction from the body part and including an end portion which is bent;
an image part to acquire an image of the end portion of the insertion part;
a work channel to form a hollow in a longitudinal direction inside the insertion part; and
an irradiating part selectively installed in the insertion part through the work channel and protruding to the end portion of the insertion part to irradiate the laser to a lateral direction.

11. The spine surgery method of claim 10, further comprising a laser blocking part to block the laser irradiated from the laser irradiating part from being irradiated to a direction in which a bent part is bent.

12. A spine surgery method comprising:
inserting a needle in a direction of an intervertebral disc through a back of a patient;
inserting a guide wire to an epidural space of the patient through the needle;
inserting an outer sheath using the guide wire;
inserting a laser surgical instrument having a bendable end portion into an inner side of the epidural space through an inside of the outer sheath using the guide wire; and
irradiating laser in the direction of the intervertebral disc.

13. The spine surgery method of claim 12, wherein in the step for inserting the laser surgical instrument into the inner side of the epidural space, the laser surgical instrument enter the inner side of the intervertebral disc in a state that the end portion of the laser surgical instrument is bent.

14. The spine surgery method of claim 13, wherein in the step for inserting the laser surgical instrument into the inner side of the epidural space, the laser surgical instrument maintains an end portion of an insertion part to be bent in a direction of a back of a patient by 10° to 40° based on a longitudinal direction of the insertion part.

15. The spine surgery method of claim 12, wherein in the step for inserting the laser surgical instrument into the inner side of the epidural space, the insertion part of the laser surgical instrument is allowed to access the outer surface of the intervertebral disc in a state that the insertion part of the laser surgical instrument maintains a straight line shape; and then, allowed to enter the inner side of the epidural space in a state that the end portion of the insertion part is bent.

16. The spine surgery method of claim 12, wherein in the step for irradiating the laser, the laser is irradiated in a direction opposite to a bent direction of the end portion of the laser surgical instrument.

17. A spine surgery method comprising:
inserting a needle into an inner side of an intervertebral disc through a back of a patient;
inserting a guide wire into the inner side of an intervertebral disc through the needle;
inserting an outer sheath using the guide wire;
inserting a laser surgical instrument into the inner side of the intervertebral disc through an inside of the outer sheath using the guide wire; and
irradiating laser to the inner side of the intervertebral disc while bending an end portion of the laser surgical instrument.

18. The spine surgery method of claim 17, further comprising injecting a dye into the inner side of the intervertebral disc while the needle is inserted into the inner side of the intervertebral disc,
wherein the step for inserting of the laser surgical instrument into the inner side of the intervertebral disc further comprises confirming a position using color information of the intervertebral disc through an image part of the laser surgical instrument.

19. The spine surgery method of claim 17, wherein in the step for irradiating the laser, the laser is irradiated in a direction opposite to a bent direction of the end portion of the laser surgical instrument.

20. The spine surgery method of claim 17, wherein the laser surgical instrument comprises:
a body part;
an insertion part extending to one direction from the body part and including an end portion which is bent;
an image part to acquire an image of the end portion of the insertion part;
a work channel to form a hollow in a longitudinal direction inside the insertion part; and
an irradiating part selectively installed in the insertion part through the work channel and protruding to the end portion of the insertion part to irradiate the laser to a lateral direction.
